(19) United States
(12) Patent Application Publication
(10) Pub. No.: US 2026/0207296 A1
(43) Pub. Date: Jul. 23, 2026

(54) GLASS-TAG FIXATION FOR MEDICAL INSTRUMENTS

(71) Applicant: Aesculap AG, Tuttlingen (DE)

(72) Inventors: Ralf Pfister, Trossingen (DE); Andre Buerk, Villingen-Schwenningen (DE); Martin Machill, Rietheim-Weilheim (DE); Maximilian Bieber, Filderstadt (DE)

(21) Appl. No.: 19/143,435

(22) PCT Filed: Dec. 19, 2023
(86) PCT No.: PCT/EP2023/086520

§ 371 Date: Jun. 26, 2025

(30) Foreign Application Priority Data
Dec. 27, 2022 (DE) 10 2022 134 821.0

(51) Int. Cl. A61B 90/98; A61B 50/00; A61B 50/30; A61B 90/00

(52) U.S. Cl. CPC A61B 90/98; A61B 50/30; A61B 90/08; A61B 2050/008; A61B 2050/0082; A61B 2050/0083; A61B 2050/0089; A61B 2090/0813

(57) ABSTRACT

A protective housing accommodates a transponder that can be received in a transponder receptacle in a medical product, such as a medical instrument and/or medical product in a sterilization circuit. The protective housing contains a first housing half and a second housing half that enclose the transponder and can be connected to one another in a form-fitting manner. The first housing half and/or the second housing half has at least one product locking geometry, via which the protective housing can be incorporated in a form-fitting manner into the medical product. The protective housing can be combined with a transponder and medical product in a system, and inserted into a medical product in a form-fitting method.

### CROSS-REFERENCE TO RELATED APPLICATIONS

This application is the United States national stage entry of International Application No. PCT/EP 2023/086520, filed on Dec. 19, 2023, and claims priority to German Application No. 10 2022 134 821.0, filed on Dec. 27, 2022. The contents of International Application No. PCT/EP 2023/086520 and German Application No. 10 2022 134 821.0 are incorporated by reference herein in their entireties.

### FIELD

The present disclosure relates to a protective housing for a glass tag, a system comprising a protective housing and a medical product, and a method for form-fittingly connecting a transponder to a medical product.

### BACKGROUND

Since the life cycle management of sterile goods is becoming increasingly important in the future, the requirement for monitoring and traceability of these sterile goods, in particular of medical/surgical instruments in a sterilization cycle, is increasing. For this reason, medical instruments of all kinds are equipped with transponders, in particular passive transponders of RFID (radio-frequency identification) or NFC (near field communication) technology. This means that the location of the medical instruments can always be identified and a statement may be made as to whether they have (correctly) passed through the entire reprocessing process or all maintenance steps. It is also possible to record the number of sterilization cycles a sterile item has undergone and to determine when the maximum service life of the sterile item has been reached. The design of the transponder in the form of a glass tag is particularly advantageous.

Devices for attaching transponders to medical instruments are already known from the prior art. For example, in document EP 2 087 850, a transponder is attached to the medical instrument, for example to its handle, with the aid of a housing, wherein the housing has a cavity for receiving the transponder. The housing may consist of two separate parts which enclose the transponder and the handle of the instrument in a recess during a fastening process and can then be form-fittingly connected. Another mounting option is to place the transponder in a hinged protective housing, which is then strapped to the medical instrument using a flexible strap/belt.

A metallic fastening element for metallic RFID tags is known from document EP 3 146 477. The fastening element is attached/glued to a medical instrument via a flat base surface and accommodates the transponder via a recess enclosed by side surfaces. The transponder and the fastening element are electrically conductively connected to each other, which can improve the signal strength or range of the transponder, in particular when attached to smaller objects.

Due to the attachment of the transponder to the outside of a medical object and the resulting protrusions, attaching the transponder according to the possibilities described above affects the handling and ergonomics of the equipped sterile goods and may also make medical reprocessing or cleaning more difficult.

In general, possibilities are also known of attaching a transponder to or in a medical instrument housing by gluing/potting/welding or in previously drilled holes. However, these solutions are complex and require appropriate preparations, such as ensuring that the surface of the medical instruments is clean and free of grease. In addition, pressing in the transponder by force-fit or gluing is not permanently durable due to the setting of plastic. Furthermore, such a connection of the transponder to the instrument makes it considerably more difficult to replace the transponder.

Often only small wall thicknesses are available to accommodate a transponder, for example a glass tag protected by a housing, in or on a medical product. This means that classic snap-on elements such as snap-on hooks may hardly be realized due to their required dimensions. In addition, certain instruments such as motor-driven saws are exposed to high vibrations during operation, meaning that these snap-on elements in small sizes are too delicate and may break in the long term.

### SUMMARY

Accordingly, the object of the present disclosure is to eliminate or at least reduce the disadvantages of the prior art.

More specifically, the object of the present disclosure is to provide a way of connecting a transponder, which may include an individual identifier of a medical tool or instrument, to the medical tool or instrument in a manner protected from environmental influences and mechanical force without changing a geometry of the instrument or tool in such a way that handling properties of the instrument or tool are adversely affected.

Specifically, the object of the present disclosure is solved by a protective housing for accommodating a transponder that can be received in a transponder receptacle in a medical product, in particular in a medical instrument and/or medicine product in a sterilization cycle, wherein the transponder receptacle is configured/provided in or by the protective housing. For this purpose, the protective housing contains a first housing half and a second housing half, which jointly enclose the transponder and are connectable/connected to each other in a form-fitting manner, wherein the first housing half and/or the second housing half has at least one product locking geometry, via which the protective housing is integrable/integrated in a form-fitting manner into the medical product.

In other words, the protective housing according to the disclosure includes the first housing half and the second housing half, which together or in cooperation with each other form the transponder receptacle. The transponder receptacle is provided and configured to receive the transponder in particular form-fittingly. The transponder is in particular a passive RFID tag of the glass tag design. However, other types of transponder that may be read wirelessly, such as an NFC tag, are also conceivable, which enable unique identification. The first housing half may be an upper housing half. The second housing half may be a lower housing half. Of course, the first housing half may also be the lower housing half and the second housing half may be the upper housing half. The terms ‘top’ and ‘bottom’ are preferably to be understood as those sides that result, for example, in the case of a rectangular body on the respective large sides. The first housing half and the second housing half are form-fittingly connectable/connected to each other and form a sandwich structure together with the transponder. The first housing half may at least partially enclose the second housing half. Furthermore, the first housing half and/or the second housing half forms or contains the product locking geometry, which enables form-fitting receiving/integrating of the protective housing in the medical product. In this context, receiving or integrating means that at least a large part of the protective housing is configured/arranged in the medical product in an assembled state and does not or only insignificantly protrudes beyond an outer contour of the medical product.

The core of the disclosure is thus the two-part protective housing, which is form-fittingly closed and contains the transponder. The two-part protective housing is thereby form-fittingly integrable into the medical product.

Such a configuration of the protective housing makes it easy to mount the protective housing. In other words, the transponder in the form of a glass tag may simply be inserted into the protective housing. In addition, the protective housing surrounds the transponder in such a way that the transponder is protected from environmental influences and mechanical damage. The product locking geometry enables tamper-proof fixing of the protective housing and thus of the transponder in the medical product without significantly changing the outer geometry of the medical product, which could adversely affect the handling properties of the medical product.

In a first aspect, the first housing half may be configured with first latching noses which may be latched into recesses configured in the second housing half.

In other words, a number of latching noses, in particular six latching noses, may be configured on the first housing half. The latching noses may be resiliently configured or resiliently attached to the first housing half. The latching noses may be configured uniformly and/or (axially) symmetrically on the first housing half, in particular in relation to a longitudinal axis of the first housing half. The latching noses may at least partially surround the second housing half and engage in the recesses of the second housing half. The second housing half may be configured with two recesses in particular.

It is explicitly pointed out that the first housing half and/or the second housing half may be configured in such a way that only a subset of the latching noses of the first housing half latch form-fittingly into the recesses of the second housing half.

A configuration with latching noses and recesses allows the form fit between the first housing half and the second housing half to be configured in a cost-effective and fail-safe manner. Furthermore, this type of configuration allows the protective housing to be assembled without tools.

In a further aspect, the latching noses of the first housing half may be operable independently of each other.

In other words, the resilient latching noses of the first housing half may be individually tensioned, relaxed or generally manipulated without this having any (mechanical) effect on neighboring latching noses.

By configuring the latching noses in this way, different functions may be mapped with the individual latching noses. Furthermore, if an individual latching nose fails, it can be ensured that the general function (e.g. the form fit between the first housing half and the second housing half and thus a protective function of the transponder) of the protective housing is maintained.

In a further aspect, the at least one product locking geometry and the first latching noses may be configured on the one spring element.

In other words, the resilient latching nose may also include or form the product locking geometry.

Synergy effects may be achieved by configuring the resilient latching noses in this way. For example, it may be achieved that the latching noses engage with the recesses of the second housing half during an insertion process of the protective housing (thereby being tensioned) and thus the first housing half and the second housing half are connected even more securely. As soon as the protective housing is inserted, the resilient latching nose can spring back and the product locking geometry can engage.

In a further aspect, the recesses of the second housing half may each be configured as a groove, wherein the groove may be configured along a longitudinal groove direction with at least one projection.

In other words, the recesses may be configured in a variable/stepped recess depth.

By configuring the recess as a groove, a planar or at least linear force application of the form-fitting connection between the first housing half and the second housing half may be achieved. By providing at least one projection or by the stepped configuration of the groove, a different contact force/type of contact/time of contact may be achieved between the individual latching noses and the recess.

In a further aspect, the first housing half may substantially correspond to the second housing half.

In other words, the first housing half and the second housing half may each be configured with latching noses and recesses. Preferably, the first housing half and the second housing half may thereby be configured with latching noses in a first portion and with recesses in a second portion, such that when the first housing half and the second housing half are brought into engagement with each other, the latching noses of the first housing half respectively come into form fit with the recesses of the second half and the latching noses of the second housing half come into form fit with the recess of the first housing half. With such a configuration of the first and second housing halves, it is conceivable that the first housing half and the second housing half differ in subordinate features, such as a curvature of an upper shell of the first housing half or second housing half.

Such an identical or almost identical design of the first housing shell and the second housing shell may reduce production costs, storage costs and tool costs.

In a further aspect, the protective housing may taper/constrict toward a connection plane between the first housing half and the second housing half.

In other words, the protective housing may have an hourglass (cross-sectional) shape, wherein a constriction point may be configured in the connection plane between the first housing half and the second housing half.

By configuring the protective housing in this way, a form fit between the medical product and the protective housing may be easily achieved when the protective housing is mounted in the medical product.

In a further aspect, the protective housing may be configured from a plastic.

In a further aspect, the protective housing may be manufactured by injection molding.

In a further aspect, the protective housing may have a substantially pill-shaped geometry.

In a further aspect, the latching nose may include a semi-circular geometry and the recess may be configured as at least two arms which are provided and configured to embrace the semi-circular geometry.

In a further aspect, the latching nose may be rigid and the recess may be resiliently configured.

In a further aspect, the protective housing may be at most twice as large as the transponder accommodated in the protective housing.

The object of the present disclosure is further solved by a system comprising the medical product, in particular medical instrument and/or medicine product in a sterilization cycle, and the protective housing. The medical product includes a protective housing receptacle provided and configured to receive the protective housing and the transponder received in the protective housing. The protective housing, in particular the protective housing according to one of the above aspects, is fixed/fixable in a form-fitting manner directly in the protective-housing receptacle of the medical product.

In other words, the system includes the medical product with the protective housing receptacle and the protective housing with the transponder. The geometry of the protective-housing receptacle is preferably adapted to a geometry of the protective housing. In other words, an internal volume of the protective-housing receptacle substantially corresponds to a volume of the protective housing. Protective housing and protective-housing receptacle are matched to each other in such a way that the protective housing is/may be fixed form-fittingly in the medical product.

In one aspect, the protective-housing receptacle may include at least one receiving groove for receiving product locking geometries of the protective housing.

In other words, the protective-housing receptacle may include the receiving groove which is provided and configured to form-fittingly receive the product locking geometry of the protective housing. The receiving groove may be configured as a substantially rectangular groove. Alternatively, the receiving groove may be configured as a groove tapering conically in a height direction.

In a further aspect, the receiving groove may be interrupted at least once in a longitudinal receiving groove direction, in a parallel portion of the protective housing receptacle.

In other words, the protective housing receptacle may contain two parallel wall portions which are connected by radius sections. The receiving groove may be interrupted in the parallel wall portions. In particular, the interruption of the receiving groove may be arranged centrally in the longitudinal direction of the receiving groove.

By interrupting the receiving groove, at least one latching nose of the protective housing may be selectively fixed/tensioned in an assembled state in such a way that the latching nose in question may be in permanent engagement with the groove of the second housing half of the protective housing, whereby the form-fitting fixation between the first housing half and the second housing half can be secured in the assembled state.

In a further aspect, the protective-housing receptacle may be configured as a passage opening in the medical product and may connect an upper side of the medical product to a lower side of the medical product. Of course, the upper side may also be an outer side and the lower side may also be an inner side of the medical product, in particular when the medical product is configured as a sterile container.

By configuring the protective housing receptacle as a passage opening, the two-part protective housing may be fixed to the medical product in such a way that the form fit between the first housing half and the second housing half only takes place when the protective housing is mounted in the medical product.

In a further aspect, the protective-housing receptacle may include a constriction between the upper side and the lower side of the medical product.

In other words, a cross-sectional area of the protective-housing receptacle may change in the height direction in such a way that the cross-sectional area, starting from the upper side, first narrows/reduces until the cross-sectional area reaches a minimum value and then widens again toward the lower side. The tapering may be configured symmetrically in relation to the height direction. In other words, the constriction may be arranged parallel to the upper side and the lower side of the medical product. In particular, a distance between the constriction and the upper side may correspond to a distance between the constriction and the lower side.

By configuring the protective-housing receptacle in this way, a form fit between the protective housing and the protective-housing receptacle may be easily configured.

In a further aspect, a center axis of the transponder receptacle of the protective housing may be configured in a plane with the constriction of the protective-housing receptacle when the protective housing is installed in the protective-housing receptacle as intended.

In a further aspect, the medical product may be configured from metal.

Furthermore, the object according to the disclosure is solved by a method for at least form-fitting connection of a transponder to a medical product, comprising the steps:
positioning the transponder in a first housing half of a protective housing;
inserting the first housing half of the protective housing into a protective-housing receptacle of the medical product from an upper side;
inserting a second housing half of the protective housing into the protective-housing receptacle of the medical product from a lower side;
form-fittingly connecting the first housing half and the second housing half.

Of course, the first housing half may also be inserted into the protective-housing receptacle from the lower side of the medical product and the second housing half may be inserted into the protective-housing receptacle from an upper side of the medical product.

Furthermore, the transponder may be positioned in the first step in the second housing half of the protective housing.

In other words, in the method, the form fit between the first housing half and the second housing half of the protective housing is only created/established when the protective housing is positioned in the protective-housing receptacle.

In yet other words, the form fit between the first housing half and the second housing half takes place simultaneously with the form fit between the protective housing and the protective housing receptacle.

In one aspect, the method may include a further step wherein the first housing half and the second housing half are firmly bonded together. This step may replace or supplement the step of the form-fitting connection. The firmly bonded connection may be performed by ultrasonic welding, hot forming, gluing or the like.

### BRIEF DESCRIPTION OF THE DRAWINGS

FIG. 1 shows a tool with a protective housing receptacle in a first embodiment;

FIG. 2 shows the protective-housing receptacle of the first embodiment in a sectional view;

FIG. 3 shows a protective housing in a first embodiment;

FIG. 4 shows the protective housing of the first embodiment in the protective-housing receptacle of the first embodiment;

FIG. 5 shows the protective housing of the first embodiment in the protective-housing receptacle of the first embodiment in a first sectional view;

FIG. 6 shows the protective housing of the first embodiment in the protective-housing receptacle of the first embodiment in a second sectional view;

FIG. 7 shows the tool with a protective housing receptacle in a second embodiment;

FIG. 8 shows the protective-housing receptacle of the second embodiment in a sectional view;

FIG. 9 shows the protective housing in a second embodiment;

FIG. 10 shows the protective housing of the second embodiment in the protective-housing receptacle in the second embodiment in a perspective view;

FIG. 11 shows the protective housing of the second embodiment in the protective-housing receptacle of the second embodiment in a first sectional view;

FIG. 12 shows the protective housing of the second embodiment in the protective-housing receptacle of the second embodiment in a second sectional view;

FIG. 13 shows a sectional view of the perspective view from FIG. 10;

FIG. 14 shows the tool with the protective housing receptacle in a third embodiment;

FIG. 15 shows the protective-housing receptacle of the third embodiment in a sectional view;

FIG. 16 shows the protective housing of a third embodiment in a first view;

FIG. 17 shows a second view of the protective housing of the third embodiment;

FIG. 18 shows the protective housing of the third embodiment in the protective-housing receptacle of the third embodiment;

FIG. 19 shows the protective housing of the third embodiment in the protective-housing receptacle of the third embodiment in a sectional view;

FIG. 20 shows the protective housing of a fourth embodiment in a first view;

FIG. 21 shows the protective housing of the fourth embodiment in a second view;

FIG. 22 shows the protective housing of the fourth embodiment in a third view;

FIG. 23 shows the protective housing of the fourth embodiment in a sectional view; and

FIG. 24 shows the protective housing of a fifth embodiment and the protective-housing receptacle of a fourth embodiment in a perspective view.

### DETAILED DESCRIPTION

Embodiments of the present disclosure are described below based on the accompanying Figures.

### First Embodiment

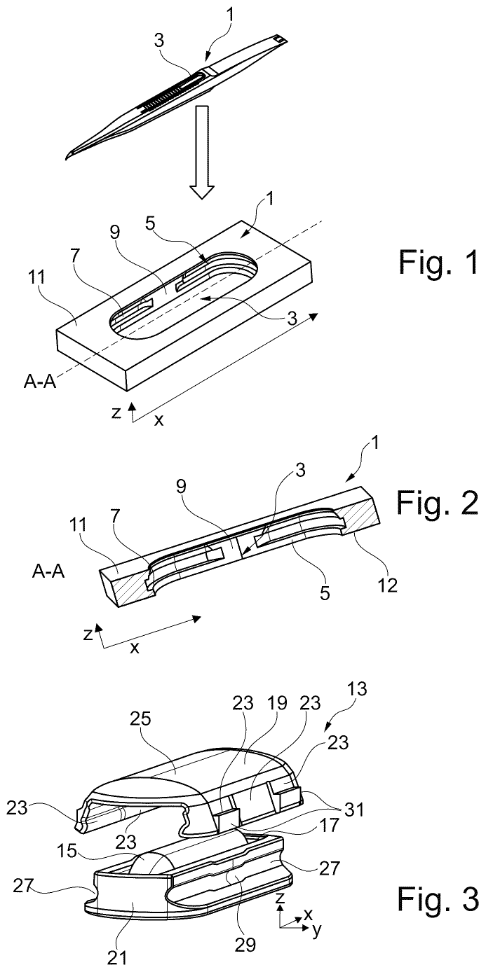

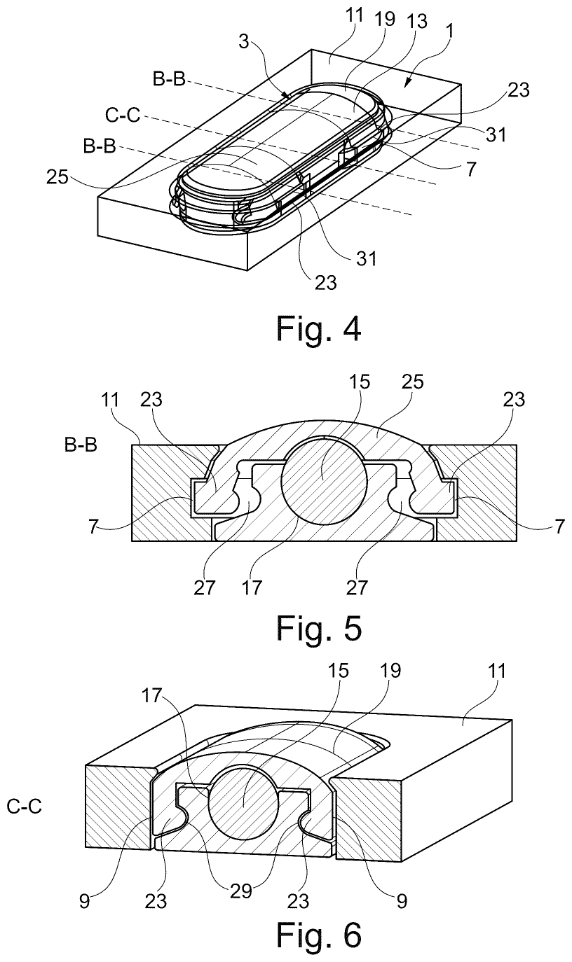

FIG. 1 shows a tool 1 with a protective-housing receptacle 3 in a first embodiment. The protective-housing receptacle 3 is shown enlarged in FIG. 1. FIG. 2 shows the protective-housing receptacle 3 in a sectional view along line A-A. The protective-housing receptacle 3 forms a substantially cylindrical clearance in the tool 1. A (negatively extruded) base surface of the protective-housing receptacle 3 is configured in a pill shape with two parallel portions and two radius sections delimiting the parallel portions. However, other geometries are also conceivable, wherein the geometry of the protective-housing receptacle 3 is matched to a geometry of the protective housing (see, for example, FIG. 4). An inner wall 5 of the protective-housing receptacle 3 contains a groove 7. The groove 7 is configured in the parallel portions and the radius portions of the protective-housing receptacle 3. In the first configuration example, the groove is configured as a substantially rectangular recess in the inner wall 5, which extends away from the protective-housing receptacle 3 or a receptacle space of the protective-housing receptacle 3. In a central portion of a longitudinal extension in the longitudinal direction X of the protective-housing receptacle 3, the groove 7 is interrupted by a projection 9. In other words, the groove 7 is not configured in the middle section. The groove 7 is configured/oriented parallel to an upper tool side 11. The groove 7 is arranged/configured substantially centrally in a height direction Z in the protective-housing receptacle 3.

In the embodiment shown here, the protective-housing receptacle 3 is configured as a continuous clearance (as a passage hole/passage opening) in the tool 1. In other words, the protective-housing receptacle 3 connects the upper tool side 11 with a lower tool side 12. However, embodiments in which the protective-housing receptacle 3 is configured as a kind of blind hole are also conceivable.

FIG. 3 shows a protective housing 13 according to the disclosure for a transponder in the form of a glass tag 15, which is accommodated in a transponder receptacle 17, in a first embodiment. The protective housing 13 substantially has a pill shape. The protective housing 13 includes a first housing half, which is the upper housing half 19 in the embodiment shown here, and a second housing half, which is the lower housing half 21 in the embodiment shown here. The transponder receptacle 17 is substantially configured in the lower housing half 21 and forms a cylindrical shape, in particular for receiving a glass tag. The upper housing half 19 includes six independent latching noses 23 extending substantially perpendicularly away from a lid portion 25 of the upper housing half 19. Three of the six independent latching noses 23 are each configured on one side of a center axis in the longitudinal direction X of the upper housing half 19. The lower housing half 21 includes a protective-housing groove 27 on opposite side portions, relative to the center axis in the longitudinal direction X, which is provided and configured to receive the latching noses 23 form-fittingly. A groove projection 29 is configured in a central portion of the protective-housing groove 27. A longitudinal extension of the groove projection 29 in the longitudinal direction X substantially corresponds to a longitudinal extension of the middle one of the three latching noses 23 in the longitudinal direction X on the corresponding side of the protective housing 13. The outer latching noses 23 as seen in the longitudinal direction X of the upper housing half 19 include tool latching hooks 31 on an outer side, which are provided and configured to engage in the groove 7 of the tool 1 and to connect the protective housing 13 form-fittingly to the tool 1. The side facing away from the glass tag 15 or the transponder receptacle 17 in an assembled state is considered to be the outer side.

FIG. 4 shows the protective housing 13 according to the disclosure of the first embodiment in an assembled state in the protective-housing receptacle 3 of the first embodiment. FIG. 5 shows a section along line B-B and FIG. 6 shows a section along line C-C. Due to the partial configuration of the groove 7 and the latching noses 23 of the protective housing 13, the six latching noses 23 are operated independently of each other and fulfill different objects/functions. The middle ones of the latching noses 23 are pressed together by the projection 9 of the groove 7 in the assembled state and thus engage in the groove projection 29 (see C-C or FIG. 6). The middle ones of the latching noses 23 thus connect the upper housing half 19 with the lower housing half 21 form-fittingly. The outer latching noses 23 configured with tool latching hooks 31 are not pressed together in the assembled state and create a form fit with the groove 7 of the protective housing receptacle 3 (see B-B in FIG. 5). The upper housing half 19 is therefore form-fittingly connected to the protective-housing receptacle 3 via the outsides of the latching noses 23 and form-fittingly connected to the lower housing half 21 via the insides of the latching noses 23.

The rectangular configuration of the groove 7 in the first embodiment creates a substantially non-detachable connection between the protective housing 13 and the tool 1. In this way, a high degree of security against accidental disassembly or manipulation of the glass tag 15 can be created. In other words, the protective housing 13 can only be removed from the tool 1 destructively, for example using a toggle press and special punches/matrices. The resulting high removal force causes the glass tag 15 to break and the protective housing 13 is severely deformed. An appropriately designed punch/die pairing can ensure that the tool itself is not damaged and that the manufacturer can fit the tool 1 with a new glass tag 15.

In the following, an assembly and insertion process of the protective housing 13 according to the first embodiment is explained by way of example with reference to FIG. 3 to FIG. 6.

In a first step, the glass tag 15 is inserted into the transponder receptacle 17 of the lower housing half 21 of the protective housing 13. The upper housing half 19 is then placed on the lower housing half 21, wherein the latching noses 23 or upper housing half 19 are brought into engagement with the protective housing groove 27 of the lower housing half 21. The latching noses 23 resiliently attached to the upper housing half 19 are substantially relaxed. When the protective housing 13 is inserted into the protective-housing receptacle 3, the latching noses 23 are pressed through the inner wall 5 into the protective-housing groove 27. This tensions the spring-mounted latching noses 23. When the protective housing 13 is arranged in a predetermined position in the protective-housing receptacle 3, the tool latching hooks 31 of the outer ones of the latching noses 23 are arranged in a plane with the groove 7 and the outer ones of the latching noses 23 can relax. By relaxing the outer ones of the latching noses 23, the form fit between the upper housing half 19 and the protective-housing receptacle 3 is established. The protrusions 9 of the protective-housing receptacle 3 prevent the inner ones of the latching noses 23, which remain in engagement with the protective-housing groove 27, from relaxing.

### Second Embodiment

The second embodiment corresponds in large parts to the first embodiment, so that the same components and features are not described again. Only the differences to the first embodiment are explained in more detail below.

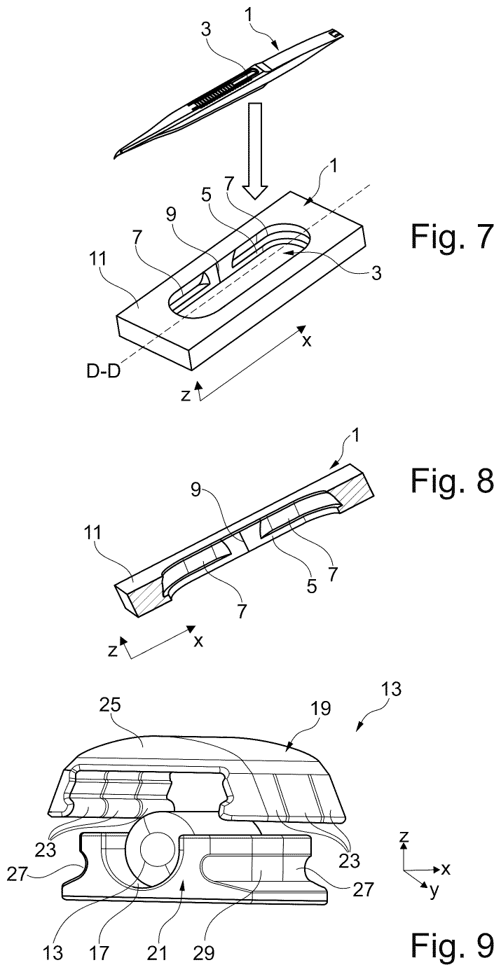

FIG. 7 shows the tool 1 with the protective-housing receptacle 3 in a second embodiment. The protective-housing receptacle 3 is shown enlarged in FIG. 7. FIG. 8 shows the protective-housing receptacle 3 in a sectional view along line D-D. In contrast to the first embodiment, the groove 7 in the second embodiment is not configured as the rectangular recess, but as a recess tapering conically in a tool height direction Z toward the upper tool side 11. In other words, in the second embodiment, the groove 7 is configured substantially without undercut.

FIG. 9 shows the protective housing 13 according to the disclosure in a second embodiment for the transponder in the form of the glass tag 15, which is accommodated in the transponder receptacle 17 of the protective housing 13, in a disassembled state. In contrast to the first embodiment, no tool latching hooks 31 are configured on the outer ones of the latching noses 23 in the second embodiment.

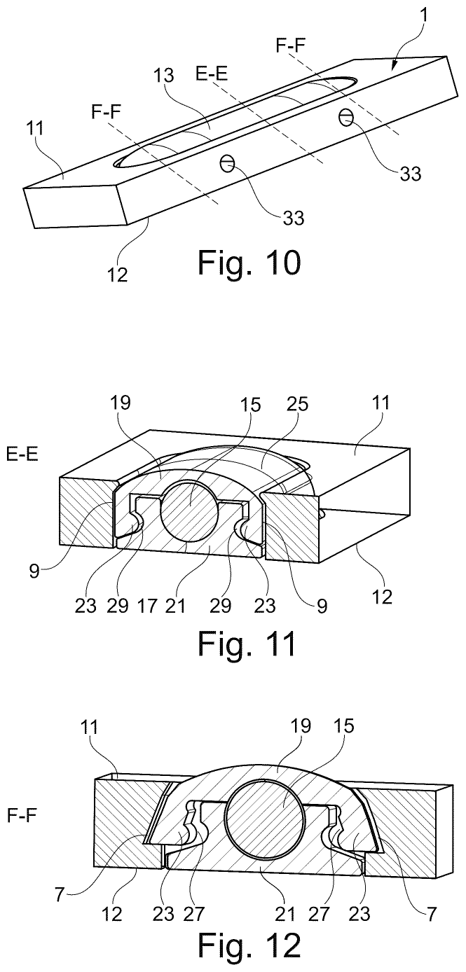

FIG. 10 shows the protective housing 13 according to the disclosure in the second embodiment in the assembled state in the protective-housing receptacle 3 of the second embodiment. FIG. 11 shows a sectional view along line E-E and FIG. 12 shows a sectional view along line F-F. The sectional view of line E-E substantially corresponds to the sectional view of line C-C of the first embodiment. In other words, also in the second embodiment, the central ones of the latching noses 23 engage in the groove projection 29 of the lower housing half 21 and thus connect the upper housing half 19 to the lower housing half 21 form-fittingly. Due to the configuration of the outer ones of the latching noses 23 without the tool latching hooks 31, the outer ones of the latching noses 23 lie in the conically configured groove 7. Furthermore, the protective-housing receptacle 3 of the second embodiment is configured with disassembly openings 33.

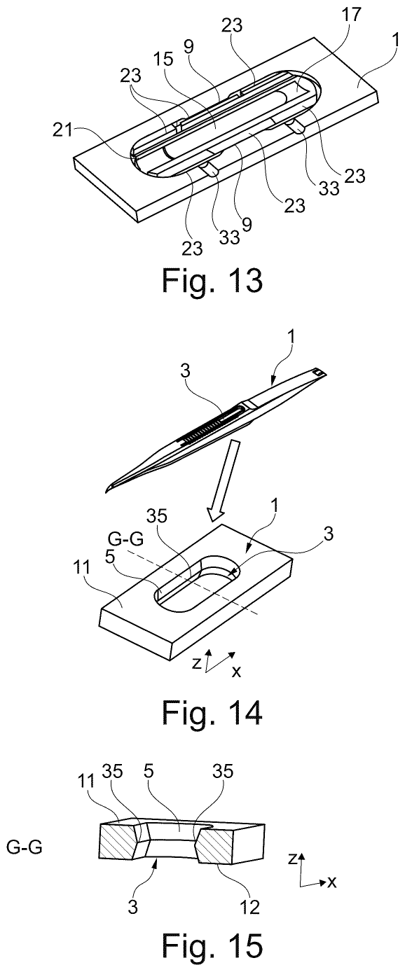

The function of the disassembly openings 33 is explained in more detail below with reference to FIG. 10 and FIG. 13.

The disassembly openings 33 are bores that are configured in a plane with the groove 7 and connect the groove 7 with an environment surrounding the tool 1. The disassembly openings 33 allow disassembly, in particular non-destructive disassembly of the protective housing 13 from the protective housing receptacle 3. For this purpose, a special tool (not shown) is brought into contact with the latching noses 23 through the disassembly openings 33. By applying a force to the special tool, the latching noses 23 can be tensioned, whereby the latching noses 23 are disengaged from the groove 7. The protective housing 13 can then be removed from the protective housing receptacle three.

Such a configuration with disassembly openings 33 is advantageous in particular in combination with the conically configured groove 7 of the second embodiment. However, embodiments are also conceivable in which the disassembly openings 33 may be configured in combination with the groove 7 of the first embodiment configured as a rectangular recess.

### Third Embodiment

The third embodiment corresponds in large parts to the first embodiment and the second embodiment, so that the same components and features are not described again. Only differences to the first embodiment and the second embodiment are explained in more detail below.

FIG. 14 shows the tool 1 with the protective-housing receptacle 3 in a third embodiment. The protective-housing receptacle 3 is shown enlarged in FIG. 14. FIG. 15 shows the protective-housing receptacle 3 in a section along the line G-G.

The protective-housing receptacle 3 of the third embodiment is configured without a groove 7. In contrast to the first embodiment and the second embodiment, the protective-housing receptacle 3 has a constriction/tapering 35. In other words, a cross-sectional area of the protective-housing receptacle 3, starting from the upper tool side 11, decreases in the height direction Z to a minimum, wherein the minimum of the cross-sectional area is reached at the constriction 35, in order to subsequently widen toward the lower tool side 12. The constriction 35 is equidistant from the upper tool side 11 and the lower tool side 12. The constriction 35 is arranged/oriented parallel to the upper tool side 11 and the lower tool side 12. In the embodiment shown here, the cross-sectional area of the protective housing receptacle 3 changes by only a few percent, by a maximum of ten percent over the height direction Z.

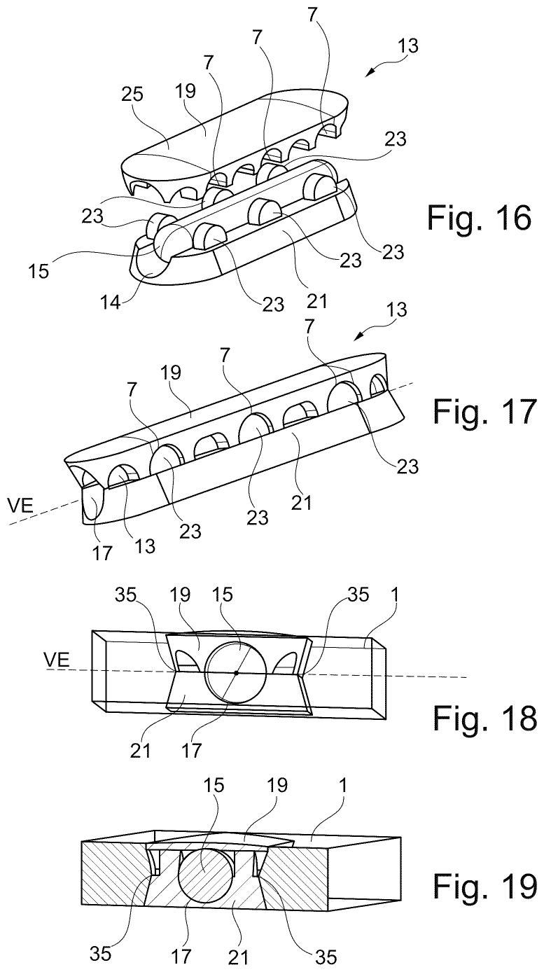

FIG. 16 shows the protective housing 13 in the third embodiment in a disassembled state.

In the third embodiment, the latching noses 23 are configured as latching circle geometries, which can be form-fittingly fixed by the grooves 7, which are configured as arms embracing the latching noses 23.

In the third embodiment shown here, the latching noses 23 are configured on the lower housing half 21 and the grooves 7 in the upper housing half 19. Of course, this is also possible the other way round.

In the third embodiment, the latching noses 23 are rigid and the grooves 7 are elastically configured.

In an alternative modification of the third embodiment, the lower housing half 21 and the upper housing half 19 may be identically configured. In yet other words, both the lower housing half 21 and the upper housing half 19 may include latching noses 23 on one side of the transponder receptacle 17 and grooves 7 on the other side of the transponder receptacle 17, so that each of the latching noses 23 meets/fits onto a groove 7. Such an assembled state is shown, for example, in FIG. 17. Each of the latching noses 23 is form-fittingly fixed by two arms, each of which forms a groove 7.

FIG. 18 shows the protective housing 13 according to the disclosure in the third embodiment in the assembled state in the protective-housing receptacle 3 of the third embodiment, and FIG. 19 shows a section through the assembled state of FIG. 18.

Both the upper housing half 19 and the lower housing half 21 have a tapered geometry. Specifically, the tapered geometry of the protective housing 13 corresponds to the constriction 35 of the protective housing receptacle 3. In other words, the upper housing half 19 and the lower housing half 21 are in flat contact in a plane VE. In the assembled state, the plane VE lies in a plane with the constriction 35. The conically tapered/conically formed geometry of protective housing 13 and protective housing receptacle 3 creates a form-fitting connection between the protective housing receptacle 3 and the protective housing 13. For this purpose, the upper housing half 19 is inserted into the protective-housing receptacle 3 from the upper tool side 11 and the lower housing half 21 is inserted into the protective-housing receptacle 3 from the lower tool side 12. The form fit between the upper housing half 19 and the lower housing half 21 takes place during the insertion process.

### Fourth Embodiment

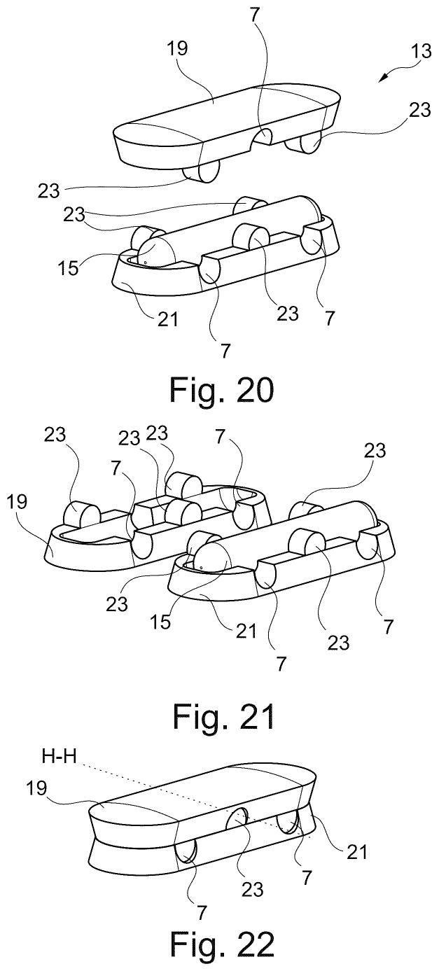

The fourth embodiment is explained in more detail below with reference to FIG. 20 to FIG. 23.

The fourth embodiment corresponds substantially to the third embodiment, so that the same components and features are not described again. Only differences between the fourth and third embodiments are explained in more detail below.

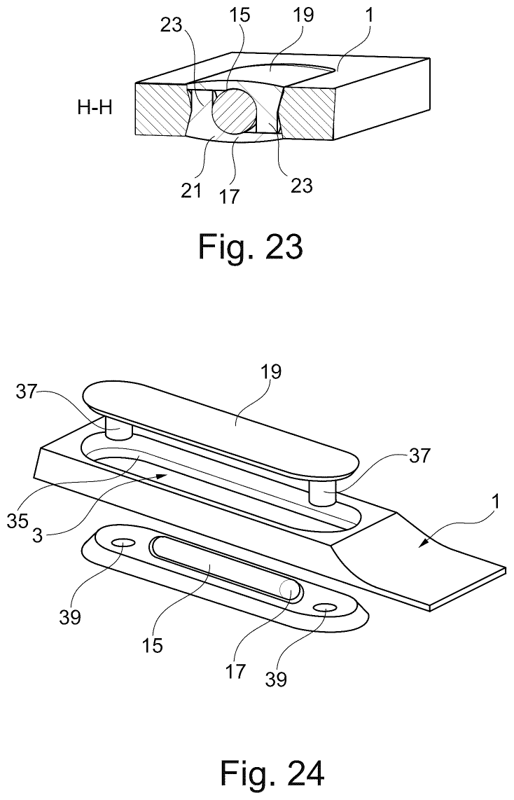

In the fourth embodiment, the upper housing half 19 corresponds to the lower housing half 21. Both the upper housing half 19 and the lower housing half 21 are configured with latching noses 23 and grooves 7, wherein the grooves 7 are configured through material recesses in the upper housing half 19 and in the lower housing half 21 respectively. The housing halves 19; 21 themselves are configured from an elastic material so that they can be latched together during assembly. The upper housing half 19 or the lower housing half 21 is configured in such a way that at each position at which a latching nose 23 is configured on one side of the housing half 19; 21, a groove 7 is configured on the other side of the housing half 19; 21 and the latching nose 23 and groove 7 alternate on the respective side of the housing half 19; 21. FIG. 20 shows the protective-housing receptacle 3 of the fourth embodiment in a disassembled state in an assembly arrangement. FIG. 21 shows the protective-housing receptacle 3 of the fourth embodiment in a state in which the upper housing half 19 is placed next to the lower housing half 21. FIG. 22 shows the protective-housing receptacle 3 of the fourth embodiment in an assembled state and FIG. 23 shows the protective-housing receptacle 3 of the fourth embodiment in a sectioned state or in a section H-H from FIG. 22.

Such an embodiment, in which the upper housing half 19 corresponds to the lower housing half 21, is particularly inexpensive to manufacture and easy to assemble (Poka Yoke).

### Fifth Embodiment

The fifth embodiment corresponds substantially to the third embodiment, so that the same components and features are not described again. In the following, only the differences between the fifth and third embodiment are explained in more detail with reference to FIG. 24.

The upper housing half 19 and the lower housing half 21 of the protective housing 13 of the fifth embodiment are configured without groove 7 and latching noses 23. The upper housing half 19 and the lower housing half 21 are joined together in the inserted state, for example by permanent deformation (hot deformation) or welding (ultrasonic welding). In the embodiment shown here, the upper housing half 19 is configured with two domes 37, which fit into corresponding recesses 39 of the lower housing half 21. The domes 37 and the recesses 39 are joined together form-fittingly in the assembled state via hot forming or ultrasonic welding.

Alternatively, the domes 37 can be glued to/into the recesses 39. Alternatively, the domes 37 can also be pressed into the recesses 39. Alternatively, for example, a screw can be screwed into the dome 37, which expands the dome 37 and fixes it in the recess 39.

### LIST OF REFERENCE SIGNS

1 tool
3 protective-housing receptacle
5 inner wall
7 groove
9 projection
11 upper tool side
12 lower tool side
13 protective housing
15 glass tag/transponder
17 transponder receptacle
19 upper housing half
21 lower housing half
23 latching nose
25 lid portion
27 protective-housing groove
29 groove projection
31 tool latching hook
33 disassembly opening
35 constriction
37 dome
39 recess
X longitudinal direction
Z height direction
VE plane

1.-14. (canceled).

15. A protective housing for accommodating a transponder configured to be received in a transponder receptacle in a medical product, the protective housing comprising:
a first housing half; and
a second housing half,
the first housing half and the second housing half configured to be connected to each other in a form-fitting manner and enclose the transponder,
at least one of the first housing half and the second housing half having at least one product locking geometry, and
the protective housing being integrable in a form-fitting manner into the medical product via the at least one product locking geometry.

16. The protective housing according to claim 15, wherein the first housing half is configured with latching noses configured to be latched into recesses configured in the second housing half.

17. The protective housing according to claim 16, wherein the latching noses of the first housing half are operable independently of each other.

18. The protective housing according to claim 16, wherein the at least one product locking geometry and the latching noses are configured on a spring element.

19. The protective housing according to claim 16, wherein the recesses of the second housing half are each configured as at least one groove, wherein the at least one groove is configured along a longitudinal groove direction with at least one projection.

20. The protective housing according to claim 15, wherein the first housing half substantially corresponds to the second housing half.

21. The protective housing according to claim 15, wherein the first housing half and the second housing half are identical.

22. The protective housing according to claim 15, wherein the protective housing tapers/constricts toward a connection plane between the first housing half and the second housing half.

23. A system comprising:
a medical product with a protective-housing receptacle;
a protective housing; and
a transponder;
the transponder being receivable in the protective housing, and
the protective housing being receivable and fixable in a form-fitting manner directly in the protective-housing receptacle.

24. The system according to claim 23, wherein the protective-housing receptacle includes a receiving groove for receiving product locking geometries of the protective housing.

25. The system according to claim 24, wherein the receiving groove is interrupted at least once in a longitudinal receiving groove direction, in a parallel portion of the protective-housing receptacle.

26. The system according to claim 23, wherein the protective-housing receptacle is configured as a passage opening in the medical product and connects an upper side of the medical product to a lower side of the medical product.

27. The system according to claim 26, wherein the protective-housing receptacle has a constriction between the upper side of the medical product and the lower side of the medical product.

28. The system according to claim 23, wherein the protective housing comprises:
a first housing half, and
a second housing half,
the first housing half and the second housing half configured to be connected to each other in a form-fitting manner and enclose the transponder,
at least one of the first housing half and the second housing half having at least one product locking geometry, and
the protective housing being integrable in a form-fitting manner into the medical product via the at least one product locking geometry.

29. A method for form-fitting connection of a transponder to a medical product, the method comprising the steps of:
positioning the transponder in a first housing half of a protective housing;
inserting the first housing half of the protective housing into a protective-housing receptacle of the medical product from an upper side of the medical product;
inserting of a second housing half of the protective housing into the protective-housing receptacle of the medical product from a lower side of the medical product; and
form-fittingly connecting the first housing half and the second housing half.
